(19) United States
(12) Patent Application Publication
(10) Pub. No.: US 2026/0314933 A1
(43) Pub. Date: Oct. 15, 2026

(54) SYSTEMS AND METHODS FOR NON-INVASIVE DETECTION OF NEURONAL FIRINGS IN HUMANS VIA QUANTUM SENSING MAGNETIC RESONANCE IMAGING

(71) Applicant: NEW YORK UNIVERSITY, New York, NY (US)

(72) Inventors: Yongxian QIAN, Rego Park, NY (US); Liz AGUILERA, Jersey City, NJ (US); Xingye CHEN, New York, NY (US); Ying-Chia LIN, Brooklyn, NY (US); Karthik LAKSHMANAN, Fair Lawn, NJ (US); Anli LIU, Brooklyn, NY (US); Yvonne W. LUI, New York, NY (US); Fernando E. BOADA, Woodside, CA (US)

(73) Assignee: NEW YORK UNIVERSITY, New York, NY (US)

(21) Appl. No.: 19/485,192

(22) PCT Filed: May 16, 2024
(86) PCT No.: PCT/US2024/029710

§ 371 Date: Nov. 17, 2025

Related U.S. Application Data
(60) Provisional application No. 63/467,482, filed on May 18, 2023.

(51) Int. Cl. A61B 5/00; A61B 5/055; G01R 33/48; G01R 33/56

(52) U.S. Cl. CPC A61B 5/0042; A61B 5/055; G01R 33/4806; G01R 33/5608

(57) ABSTRACT

A system for detection of neuronal firings using quantum-sensing magnetic resonance imaging (MRI) includes an MRI scanner programmed to execute a first pulse sequence for imaging and a second pulse sequence for free induction decay (FID) signal acquisition. The system includes a multi-channel array coil operatively coupled to the MRI scanner. The multi-channel array coil includes a plurality of coil elements, each coil element corresponding to a separate coil channel and a quantum sensing volume. The system includes a processor and a memory storing instructions that when executed by the processor, cause the processor to receive FID signals from each coil channel. The FID signals include a time series of samples separated by a sample time interval. The instructions further cause the processor to determine neuronal magnetic fields induced by neuronal firings at each coil element, based on the FID signals, and display the neuronal magnetic fields.

### CROSS-REFERENCE TO RELATED APPLICATIONS

This application claims the benefit of priority to U.S. Provisional Application No. 63/467,482, filed May 18, 2023, the entirety of which is incorporated herein by reference.

### STATEMENT OF GOVERNMENT INTEREST

This invention was made with government support under AG067502 awarded by the National Institutes of Health. The government has certain rights in the invention.

### TECHNICAL FIELD

The present disclosure relates generally to systems and methods for non-invasive detection of neuronal firings in human via quantum-sensing magnetic resonance imaging (qsMRI).

### BACKGROUND

Cognitive function is physically executed by neuronal electrophysiological activities (called firings) that activate action potentials and conduct them along axons and across synapses towards other neurons. These firings occur in a circuit of specialized cognitive function, such as perception, attention, memory, language, etc. Aging and diseases may impair cognitive functioning, such as memory loss, difficulties with wording, and poor decision-making, as seen in the populations of normal aging, mild cognitive impairment (“MCI”), and Alzheimer's disease (“AD”). Therefore, detection of neuronal firings is crucial to assessment of cognitive impairment and other neurological disorders.

By the nature of neuronal firings being electrical signals within the skull, there is a focus on non-invasive rather than invasive approaches. Current approaches to non-invasive detection of neuronal firings in the human brain are limited to electroencephalography (“EEG”), magnetoencephalography (“MEG”), and functional magnetic resonance imaging (“fMRI”). Signals from EEG and MEG have substantially contributed to our understanding of an array of neurological disorders such as epilepsy, brain injury, and cognitive impairment. However, scalp EEG and MEG, although non-invasive, detect neuronal fields relatively far away (~20 mm) from firing sources and have difficulty to locate firing sources, and thus limit their sensitivity to slow-changing, easily-synchronized postsynaptic currents in a relatively large brain region. fMRI, as another non-invasive technique, detects brain regional connectivity (network) at resting state (without external stimuli), and explores circuitry of cognitive functions at task-activated state (with specialized external stimuli). These fMRI techniques, while opening tremendous opportunities for human cognition studies, share two major technical drawbacks. One is the indirect detection of neuronal activities via blood oxygen level dependent (“BOLD”) signal which has a complex and unknown relationship with neuronal activities. The other is the relatively low temporal resolution (40-100 ms) which is unlikely able to detect fast-changing action potentials (~2 ms in duration) and postsynaptic currents (~40 ms in duration) during neuronal firings. To overcome these technical drawbacks, a complementary combination of fMRI (of high spatial resolution ~4 mm) with scalp EEG or MEG (of high temporal resolution ~2 ms) has been implemented by our group and others in recent years. But it has intrinsic technical difficulties such as severe signal interference between EEG and fMRI, and low acceptability (comfort) to study participants. Another strategy was recently proposed to easy this technical difficulty, in which the oscillating brain waves, instead of action potential spikes, may be detected using double resonance idea via spin-lock techniques. This type of techniques has not yet fully tested on human studies. Also recently proposed is the high temporal resolution (milliseconds or even sub-milliseconds) fMRI, under the assumption that neuronal activity is exactly repeatable in time, though unlikely to be practical.

While existing techniques provide valuable tools to study and monitor neuronal firings for research and clinical purposes, they exhibit drawbacks that leave a need for methods that allow for the detection of relatively weak electrical signals in a non-invasive manner and for a temporal resolution such that very short duration changes can be detected.

### SUMMARY

One embodiment relates to a system for detection of neuronal firings using magnetic resonance imaging (MRI) including an MRI scanner. The MRI scanner is programmed to execute a first pulse sequence configured for imaging and a second pulse sequence configured for free induction decay (FID) signal acquisition. A multi-channel array coil is operatively coupled to the MRI scanner. The multi-channel array coil includes a plurality of coil elements. Each coil element in the plurality of coil elements corresponds to a separate coil channel and a sensing volume. The system further includes a processor and a memory. The memory stores instructions executable by the processor. When executed by the processor, the instructions cause the processor to receive FID signals generated by the MRI scanner from each coil channel. The FID signals include a time series of samples separated by a sampling time interval. The instructions further cause the processor to receive FID signals generated by the MRI scanner from each coil channel, the FID signals comprising a time series of samples separated by a sample time interval. The instructions further cause the processor to determine neuronal magnetic fields induced by neuronal firings at each coil element, based on the FID signals. The instructions further cause the processor to display the neuronal magnetic fields.

One embodiment relates to a method of detecting neuronal firings in a subject with a magnetic resonance imaging (MRI) machine. The method includes positioning multi-channel array coils on the subject, the multi-channel array coils comprising a plurality of coil elements. The method includes executing an imaging pulse sequence, using the MRI machine, to acquire MRI images from each coil element in the plurality of coil elements, each coil element corresponding to a local neuronal firing volume. The method includes executing a free induction decay (FID) signal acquisition pulse sequence, using the MRI machine, to acquire FID signals from each coil element. The method includes determining neuronal magnetic fields at each coil element using the FID signals based on a phase difference between neighboring samples of the FID signals. The method includes locating neuronal firing sources using the acquired MRI images from each coil element, by: sensing neuronal firings using proton nuclear spins in water molecules inside neurons as endogenous quantum sensors. The method includes modulating neuronal magnetic fields onto the main magnetic field of the MRI machine. The method includes recording neuronal firings, using the MRI machine, via the acquired FID signals from each coil element.

One embodiment relates to a computer-readable medium storing instructions, which when executed by a processor, causes the processor to receive FID signals generated by an MRI machine. The FID signals include a time series of samples separated by a sampling time interval. The instructions further cause the processor to receive MRI images generated by the MRI machine of a sensing region. The instructions further cause the processor to determine neuronal magnetic fields, using the FID signals, by determining a phase difference between neighboring samples of the FID signal locate neuronal firing sources, using the MRI images, by utilizing nuclear spins of hydrogen protons inside neurons as quantum sensors to detect neuronal firing locally, determining a phase difference at the sampling time interval, and determining neuronal magnetic fields using the phase difference. The instructions further cause the processor to display the neuronal magnetic fields with time-varying series and firing-source images.

### BRIEF DESCRIPTION OF THE FIGURES

Before turning to the figures, which illustrate certain embodiments in detail, it should be understood that the present disclosure is not limited to the details or methodology set forth in the description or illustrated in the figures. It should also be understood that the terminology used herein is for the purpose of description only and should not be regarded as limiting.

FIGS. 1A-1C show a system for quantum-sensing magnetic resonance imaging (qsMRI) of local neuronal firings, according to an example embodiment.

FIGS. 2A-2C show a conceptual schematic of qsMRI, according to an example embodiment.

FIGS. 3A-3B show the localization of qsMRI for neuronal firing sources in the human brain and the free induction decay (FID) signals from each of the firing source regions.

FIG. 4 is a flowchart for the calculation of neuronal firing magnetic field B_{n,z}.

FIGS. 5A-5B show the display of neuronal firing sources and neuronal magnetic fields, according to an example embodiment.

FIGS. 6A-6C show computer simulation results showing the measurement accuracy of qsMRI.

FIGS. 7A-7C show results of a phantom study.

FIGS. 8A-8B show results of a study conducted with a healthy subject (25-year-old female).

FIGS. 9A-9B show results of a study conducted with a healthy subject (25-year-old male).

FIGS. 10A-10C show neuronal firings from cognitively normal subjects for resting-state MRI scans at early morning.

FIG. 11 shows a summary of neuronal firings in a 100 ms-long window of FID signals from a group of 13 cognitively normal subjects for resting-state MRI scans at early morning.

FIGS. 12A-12C show neuronal firings from the brain of a 43-year-old healthy female subject acquired with a 20-channel Head/Neck coil at 3 T, with and without a finger-tapping task.

FIGS. 13A-13C show neuronal firings from the brain of a 74-year-old healthy male subject acquired with a 20-channel Head/Neck coil at 3 T, with and without a finger-tapping task.

FIGS. 14A-14D shows a summary of neuronal firing rates and the changes in firing rate, with and without a finger-tapping task.

FIG. 15 shows a schematic of the system for qsMRI of local neuronal firings of FIGS. 1A-1C.

Reference is made to the accompanying drawings throughout the following detailed description. In the drawings, similar symbols typically identify similar components, unless context dictates otherwise. The illustrative implementations described in the detailed description, drawings, and claims are not meant to be limiting. Other implementations may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

### DETAILED DESCRIPTION

Embodiments described herein relate to systems and methods for non-invasive detection of neuronal firings (action potentials and postsynaptic potentials) via quantum-sensing magnetic resonance imaging, referred to herein as quantum-sensing MRI (“qsMRI”). This qsMRI technique employs localized magnetic resonance (“MR”) signal (called free induction decay or “FID”) to achieve a high temporal resolution (e.g., 0.25-0.01, etc.) more than 10 times that in scalp electroencephalogram (EEG) recordings, without the need for electrodes. Therefore, the qsMRI technique will have a wide range of scientific and clinical applications to better understanding of cognitive functions and neuronal firings in the aging, MCI, and AD brains, as well as other neurological disorders such as epilepsy, traumatic brain injury, multiple sclerosis, brain tumors, cognitive impairment, and dementia.

### Overview

qsMRI provides three major features that provide advantages over existing techniques. First, the qsMRI technique is able to detect extremely weak magnetic fields (<10⁻⁶ μT on the skull) induced by neuronal firings (action potentials and postsynaptic currents). This feature is accomplished through the use of proton (¹H) nuclear spins in water molecules inside neurons (including soma, axon, and dendrites) as endogenous quantum sensors to detect neuronal firings locally. This technique for detection is also accurate due to the phase evolution with time of these quantum sensors. A second feature is the ability to detect very fast-changing action potentials (e.g., 2 ms in duration) and postsynaptic currents (e.g., 40 ms in duration) during neuronal firings. The qsMRI technique leverages an existing MRI system to acquire FID signals at a very high temporal resolution (e.g., 0.25-0.01 ms), depending on a dynamic range of the quantum sensors to be used. A third feature is the ability to localize sources of neuronal firings in the brain and/or other body regions. The qsMRI technique takes advantage of localized sensitivity of individual coil elements to definitively determine which brain or body regions the neuronal firings come from.

### System for Detecting Neuronal Firings

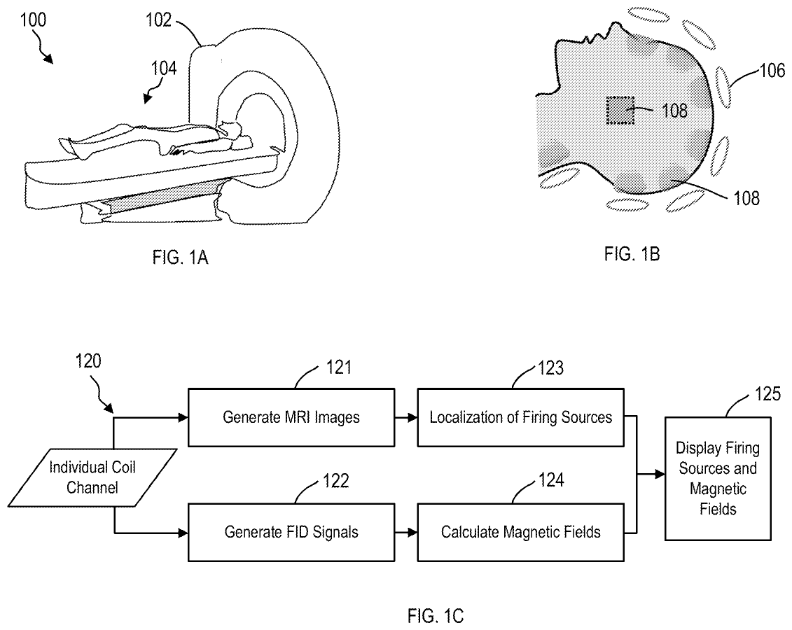

Referring generally to FIGS. 1A-1B, in one embodiment, a qsMRI system 100 for non-invasive detection of neuronal firings in the human brain and body comprises an MRI machine 102 (e.g., system, device, scanner, etc.) and two pulse sequences 104 executed on the MRI machine 102. A first pulse sequence is configured for MRI imaging and the second pulse sequence is configured for FID signal acquisition. Therefore, the MRI machine 102 is able to generate MRI images and acquire FID signals. In some embodiments, the pulse sequence for FID signal acquisition is able to perform selective and/or non-selective RF excitation. In some embodiments, the MRI machine 102 may be a typical clinical device, including low field (<1 T) MRI machines such as those considered portable or desktop. The MRI machine 102 may be connected (via cables, connectors, ports, wireless connectivity, etc.) to a radiofrequency (RF) coil 106 or an array of RF coils. In some embodiments, the RF coils 106 are multi-channel receive coils (e.g., head coils, spine coils, body coils, etc.) that allow for simultaneous reception of MR signals from multiple spatial locations. In some embodiments, the RF coils 106 are transceiver coils. The more coil channels the higher the spatial resolution will be. RF coils 106 correspond to coil channels through which MRI and FID signals are received. In some embodiments, each RF coil 106 corresponds to one coil channel. In some embodiments, each RF coil corresponds to a plurality of coil channels. In some embodiments, sensing volumes 108 (e.g., imaging volume), are passively defined by a sensitivity profile of an RF coil 106, as indicated by the shaded portions. Sensitivity profiles are typically characterized by coil geometry and proximity to the imaged anatomy. In some embodiments, sensing volumes 108 are actively defined by selective excitation of RF pulses. In some embodiments, the qsMRI system 100 may utilize two separate calculations, which may be performed by separate software programs or merged into a single program, one for calculation of the magnetic field induced by neuronal firings from FID signal and the other for display of the calculated neuronal magnetic fields. The calculated neuronal magnetic fields may be used to localize neuronal firing sources in the brain.

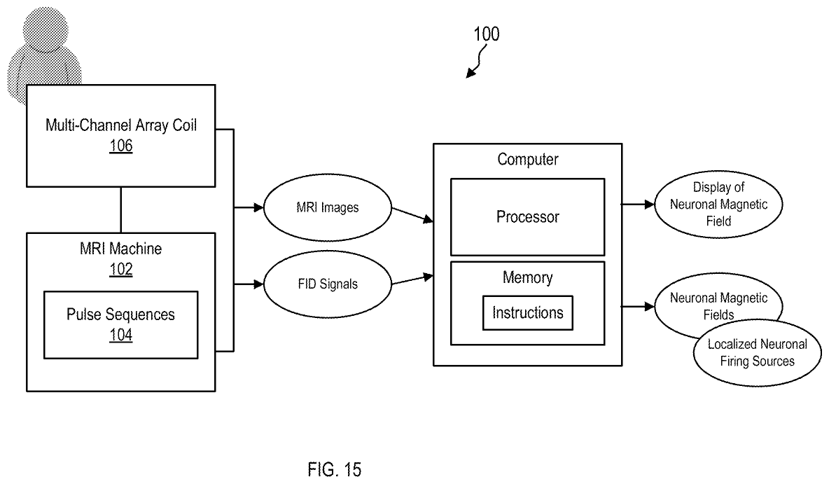

FIG. 1C is a flow chart of a method 120 occurring within the qsMRI system 100 for each individual coil channel in the qsMRI system 100, and FIG. 15 shows a schematic of the qsMRI system 100. At step 121, MRI images are generated from data acquired through the individual coil channel. At step 122, FID signals are acquired from the individual coil channel. At step 123, neuronal firing sources are localized using the MRI images generated at step 121. Typically, localizing neuronal firing sources directly using MRI images is challenging due to limited temporal resolution. However, the temporal resolution provided by the qsMRI system 100 is much higher in comparison. At step 124, neuronal magnetic fields are calculated at each individual channel using the FID signals generated in step 122. At step 125, the neuronal firing sources and neuronal magnetic fields are displayed.

Displaying the calculated neuronal magnetic fields provides a visualization of the spatial distribution and temporal dynamics of neural activity in the brain. In some embodiments, displaying calculated neuronal magnetic fields can aid in the localization of abnormal neural activity associated with neurological disorders (epilepsy, stroke, or brain tumors, etc.) so regions of aberrant magnetic field patterns may be identified. In some embodiments, displaying calculated neuronal magnetic fields can be used to validate computational models, investigate functional connectivity, or predict functional outcomes.

### Method for Detecting Neuronal Firings

In one embodiment, a method for detecting neuronal firings includes three algorithms. The first algorithm is used to calculate neuronal magnetic fields from acquired FID signals by taking a phase difference between neighboring samples in the FID signals. The second algorithm is used to locate neuronal firing sources by taking MRI images from individual coil channels. Localization of neuronal firing sources refers to the process of determining the spatial origin or location within the brain where neuronal firing originates. In some embodiments, localization of neuronal firing sources is used in understanding brain function, mapping neural circuits, and/or diagnosing neurological disorders. The third algorithm is used to display/present the calculated neuronal magnetic fields with time-varying series and display neuronal firing source images. Displaying neuronal firing source images can provide spatial information that maybe used in the localization of neural activity. In some embodiments, displaying neuronal firing source images can be used to understand brain function, investigate disease, or evaluate treatment.

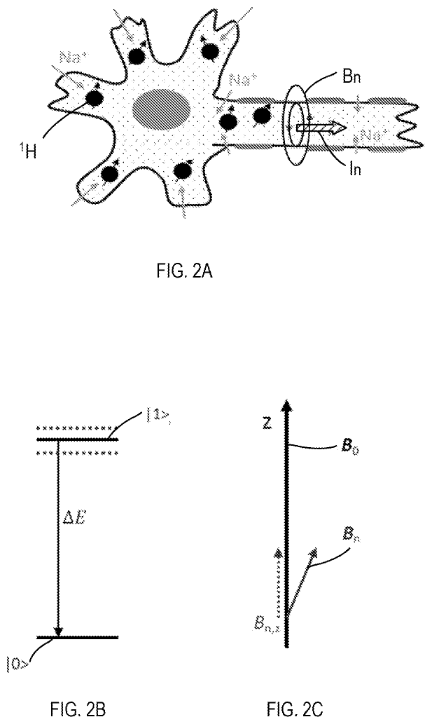

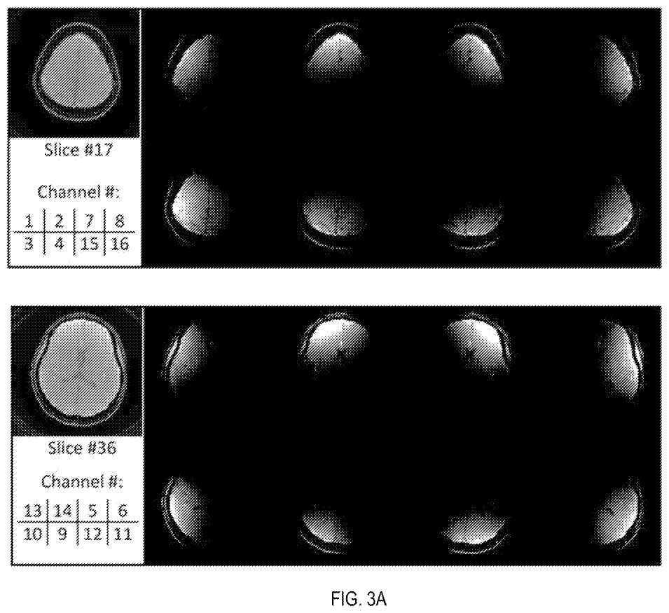

Referring to FIGS. 2A-2C, aspects of calculating neuronal magnetic fields from acquired FID signals are shown. FIG. 2A shows quantum sensors of endogenous proton (¹H) nuclear spins (black dots with arrows) of water molecules which are immersed in sodium (Na⁺) ionic flow (neuronal electrical currents caused by action potentials and/or postsynaptic potentials) in a firing neuron. In addition, a neuronal electric current Iₙ, and the a neuronal firing magnetic field Bₙ generated by the neuronal electric current Iₙ are shown. Quantum sensors may be leveraged to enhance sensitivity and resolution in MRI. qsMRI utilizes the nuclear spins of hydrogen protons inside neurons as quantum sensors to detect and measure magnetic fields with high sensitivity and precision. In particular, quantum sensors are suitable for the detection of weak magnetic field strengths generated by neuronal activity. As such, quantum sensors can improve the detection of neuronal firing. FIG. 2B shows how quantum sensors sense magnetic fields induced by neurons during firing. In particular, FIG. 2B shows quantum sensing of time-varying neuronal magnetic field B_{n,z}(t) by proton spins transiting from the excited quantum state |1> to ground state |0>, with an energy difference ΔE that changes with the neuronal magnetic field around a static value defined by the main magnetic field B₀ of an MRI machine, such as MRI machine 102. FIG. 2C shows modulation of neuronal firing magnetic field Bₙ onto the main magnetic field B₀. The modulation is naturally encoded in the phase of the FID signals.

The qsMRI method can utilize the local endogenous nuclear spins of the hydrogen protons inside neurons as quantum sensors, each with states |0> and |1> in the static external magnetic field B₀. The target magnetic field Bₙ(r, t), induced by electric currents {Iₙ(r, t), r in ΔV} is measured at location r and time t, such as at a location in the brain. The electric currents can be generated by firing neurons in axons and in close proximity to the location. The sensing process then proceeds through electromagnetic interaction of these nuclear spins at a superposition state (achieved through the exciting RF pulse) with the target magnetic field Bₙ. During the sensing period, the nuclear spins accumulate an extra phase φ(r, t) as set in Eq. 1 below. Phase φ(r, t) at location r and time t is caused by the neuronal magnetic field B_{n,z}(r, t) That extra phase is encoded into the FID, s(t), in Eq. 2. FID signal s(t), at a quantum-sensing volume ΔV is defined by coil sensitivity c(r), magnetization density ρ(r), and relaxation time constant

$${T}_{2}^{*} ( r ) .$$

The spin states are read-out by RF coils placed around a body part, such as the head, during data acquisition of the FID signal from the spin ensemble in a coil specific investigation volume ΔV of magnetization density ρ(r) and effective transverse relaxation constant

$${T}_{2}^{*} ( r ) .$$

A phase difference at a sampling interval Δt is calculated for an estimate of the firing field z-component B_{n,z}, in Eq. 3. The firing field B_{n,z} is estimated via phase difference of the FID signal at a sampling interval Δt. While the firing field is tiny in one embodiment (around ~0.1 nT) above the scalp, the field is large enough (~18-363 μT) inside the axons (~0.2-2.0 μm diameter) in the brain to generate a detectable change in the local resonance frequency. This change in resonance frequency may be on the order of 15.5 kHz for proton MRI.

$$φ ( r , t ) = φ ( r , TE ) + γ {\int }_{TE}^{t} {B}_{n , z} ( r , τ ) d τ \qquad Eq . 1$$

$$s ( t ) = {\int }_{Δ V} c ( r ) ρ ( r ) {e}^{- t / {T}_{2}^{*} ( r )} {e}^{- j φ ( r , t )} dr \qquad Eq . 2$$

$$\begin{matrix}\text{?} ( t ) = ( 1 / γ ) d φ / dt \approx ( 1 / γ ) \{ phase [ s ( t ) s * ( t + Δ t ) ] \} / Δ t & Eq . 3\end{matrix}$$

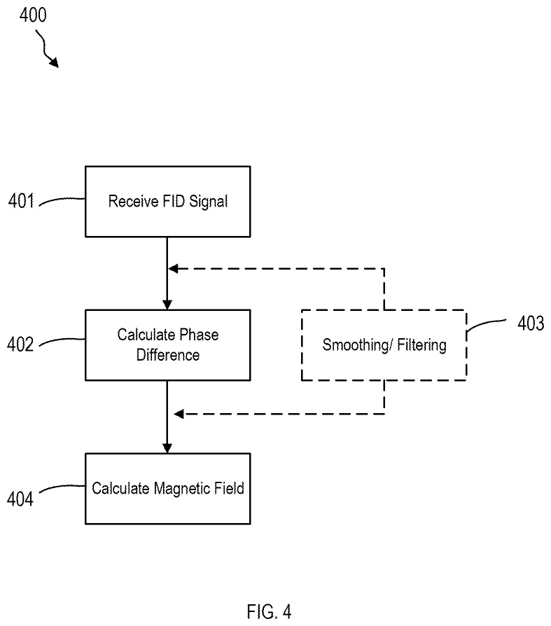

FIG. 4 shows flowchart for a method 400 of calculating a neuronal firing magnetic field B_{n,z}. At step 401, FID signals, from individual coil channels are received (Eq. 4). FID signals are complex-valued time series with a sampling time interval Δt. At step 402, a phase difference Δφ between neighboring (±Δt) samples is calculated by taking the phase of conjugate production between the two samples, as set in Eq. 5. In this way, the phase wrap-around effect on Δφ can be effectively minimized if sampling interval Δt is small enough, i.e., Δt<π/(γBₘₐₓ), where Bₘₐₓ is the dynamic range of qsMRI measurements for neuronal magnetic field B_{n,z} estimated from the finite difference of phase, Δφ/Δt, with a gyromagnetic ratio γ. At step 404, neuronal magnetic fields are calculated, as set in Eq. 6. Optionally, at step 403, three-point smoothing or low-pass filtering may be used to reduce random noise interference on the phase calculation of FID signals and/or on the estimation of neuronal firing magnetic fields.

s(t)=a(t)e^{iφ(t)} (Eq. 4

Δφ(t)=phase(s(t±Δt)s*t)) Eq. 5

B_{n,z}(t)=(1/γ)Δφ/Δt Eq. 6

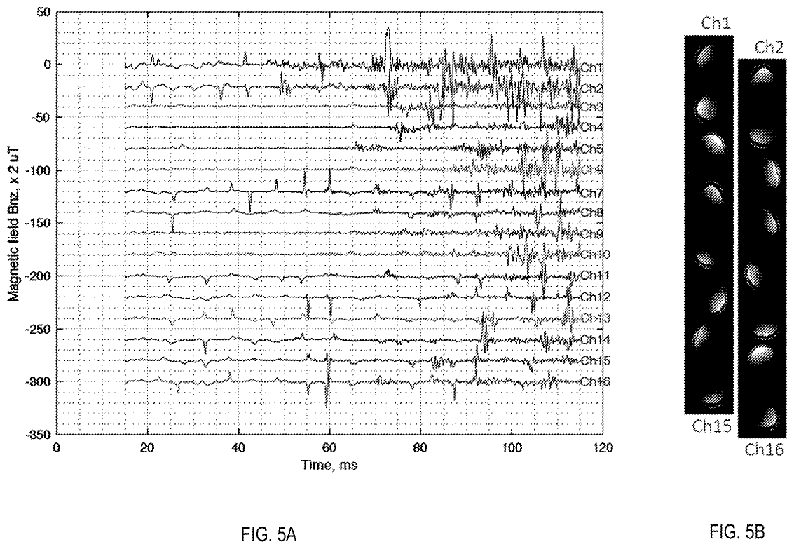

Referring to FIGS. 5A-5B, an example display of neuronal firing sources and neuronal magnetic fields is shown. FIG. 5A is a display of neuronal firing magnetic fields, B_{n,z}(t), in rows in the order of coil channel index, with a vertical scale for magnetic strength and a horizontal scale for time. This panel displays neuronal magnetic field for single or multiple FID signals at a time and can be refreshed (real-time or off-line) for the next FID readout. FIG. 5B is a display of firing sources (images at individual coil channels) around the display panel in FIG. 5A: top, bottom, left, or right.

These system components and other aspects of the present invention will become apparent to those skilled in the art after a reading of the following detailed description of the invention, including the figures and appended claims.

### Supporting Results

The following section describes studies relating to qsMRI. These examples are merely illustrations and should not be construed as limiting the disclosure.

### Localization of Neuronal Firing Sources in the Human Brain

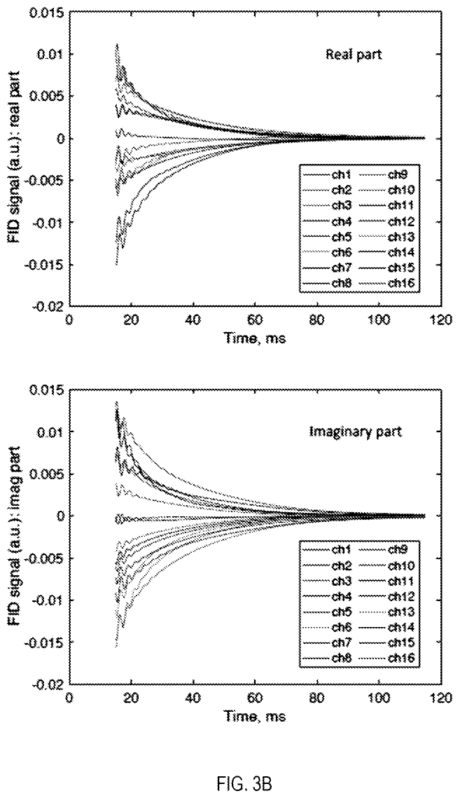

FIGS. 3A-3B illustrate examples and results relating to localization of qsMRI for neuronal firing sources in the human brain, which is determined definitely by MRI images from individual coil channels in a multi-channel receive coil array.

Method: Healthy subjects (n=4, age 28.0±5.4, 3/1 male/female). MRI Scanner: 3 T Prisma (Siemens). Coil: Siemens Head/Neck 20Ch coil (16 channels were used for the head). Sequence: vendor's product sequence AdjFreq. Data Acquisition: proton (1H) FID at resting state, readout time 100 ms (sampling interval=0.0997 ms), TE=15 ms, and flip angle=10°. Spatial Localization: individual coil-channel images. Data processing: a custom-developed program in MATLAB R2021a (MathWorks, Natick, MA), including 3-point smoothing before and after the phase calculation in Eq. 3. Computer Simulation: to determine accuracy of the qsMRI measurements for action potentials and postsynaptic potentials via Eqs. 1-3 above.

FIG. 3A shows MRI images (left: combined; right: uncombined) of sensing volumes defined by two subgroups of eight coil elements at the top (slice #17) and middle (slice #36) levels of a brain. FIG. 3B shows FID signals (real and imaginary) from the individual sensing volumes at the sixteen coil elements shown in FIG. 3A.

### Computer Simulations

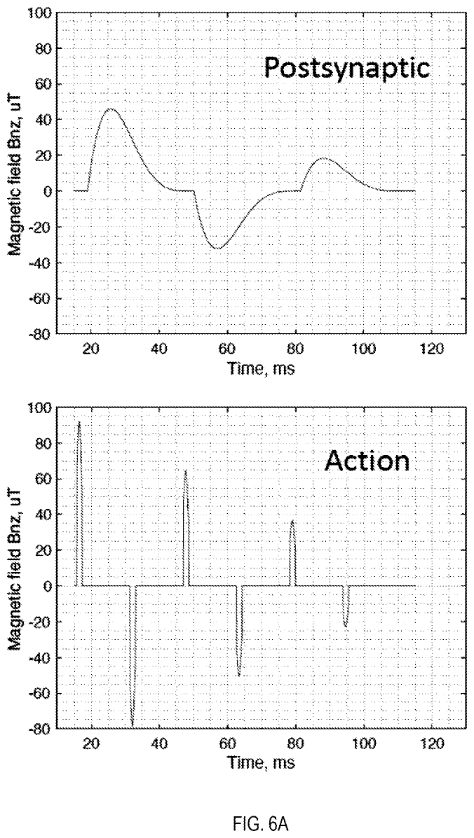

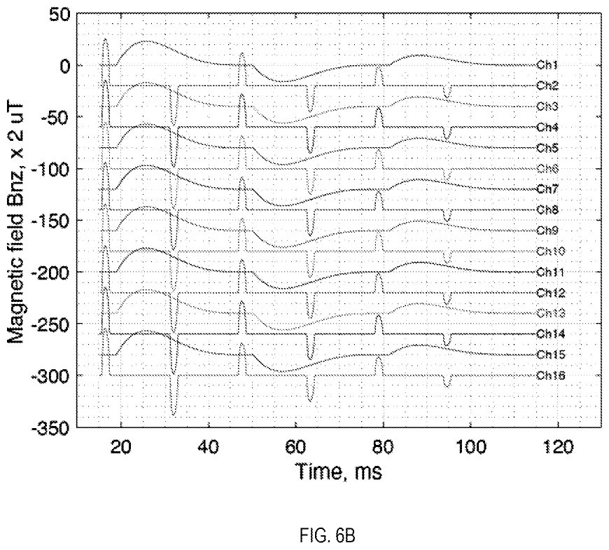

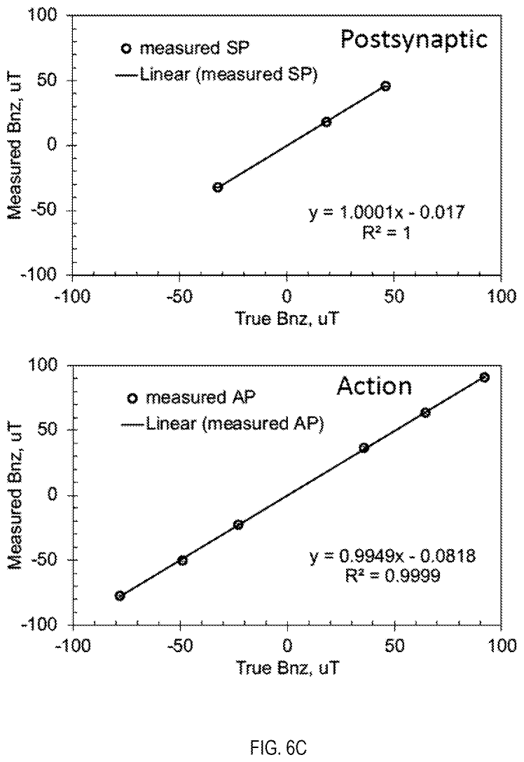

FIGS. 6A-6C illustrate results relating to computer simulations used to determine the measurement accuracy of qsMRI.

FIG. 6A shows computer models of neuronal magnetic field B_{nz} induced by the postsynaptic potentials (40 ms-long for each firing) and action potentials (2 ms-long for each firing). FIG. 6B shows qsMRI recordings calculated from the simulated FID signals (sampling interval=0.0977 ms, SNR=250, additive Gaussian random noise independent across channels). FIG. 6C is a quantitative comparison of the amplitude of peaks between the qsMRI measurements in FIG. 6B and true values in FIG. 6A. The relative error of qsMRI measurements is <0.7% for the action potentials and <1.9% for the postsynaptic potentials. The error bars in FIG. 6C are smaller than the size of markers and barely visible. Therefore, the measurement accuracy of qsMRI is demonstrated.

### Phantom Study

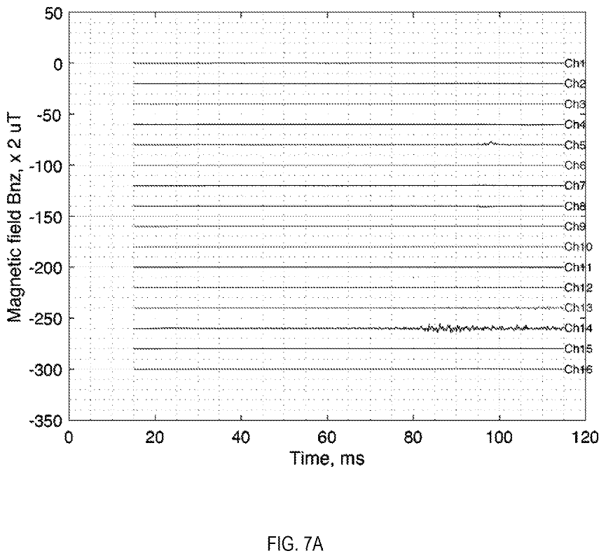

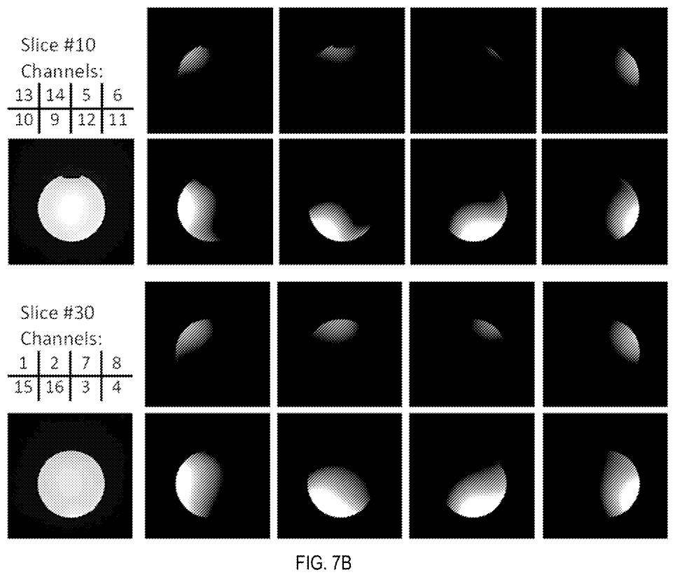

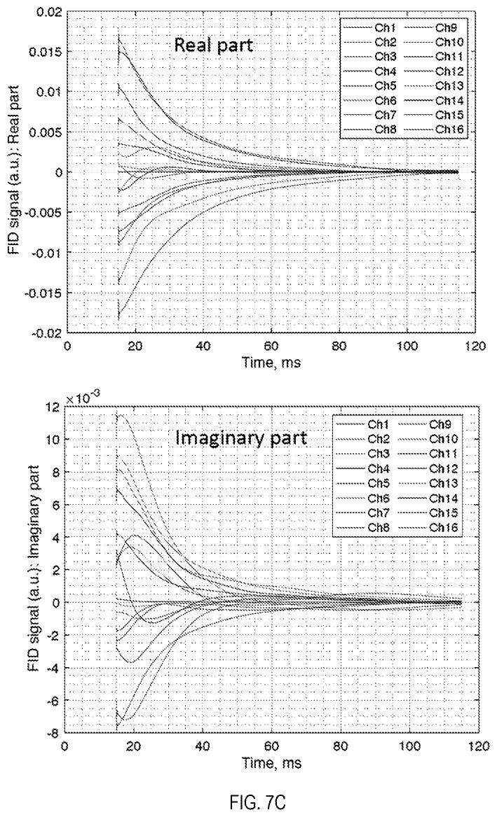

FIGS. 7A-7C illustrate results relating to a phantom study confirming that there is no qsMRI signal when there is no neuronal firing. The phantom is a plastic bottle (2000 ml) filled with saline water (0.5% ww in NaCl). The study was performed on a clinical MRI scanner at 3 T (Siemens Prisma and 20-channel Head/Neck coil).

FIG. 7A shows the qsMRI recordings from each of the 16 coil channels. FIG. 7B shows MRI images of the phantom showing the sensing volumes at individual channels. FIG. 7C shows FID signals (real and imaginary) that were used to calculate the qsMRI recordings in FIG. 7A. Note: the display window/level in FIG. 7B are the same for all the uncombined images.

### Study on Healthy Subjects

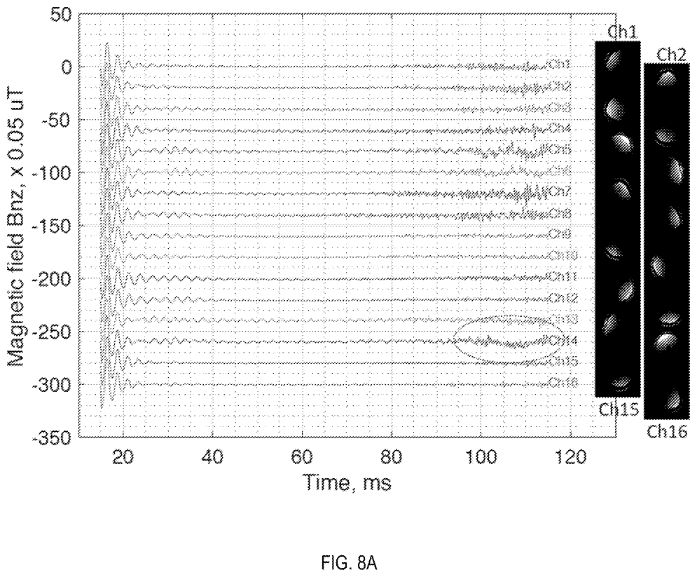

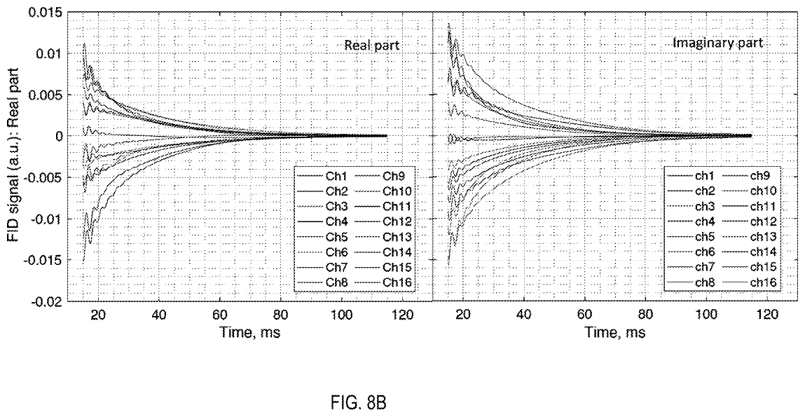

FIGS. 8A-11 illustrate results relating to studies performed on healthy subjects. The studies were performed on a clinical MRI scanner at 3 T (Siemens Prisma and 20-channel Head/Neck coil).

FIGS. 8A-8B show results relating to a study on a healthy subject (25-year-old female). FIG. 8A shows qsMRI recordings at resting state and associated sensing regions in the brain (MRI images are attached to each channel). FIG. 8B shows FID signals (real and imaginary) that were transformed into the qsMRI recordings in FIG. 8A. A postsynaptic signal (~20 ms in duration, dashed circle) is visible at Channel #14 (left-side prefrontal lobe). The waves at the beginning period of time (15-40 ms) in FIG. 8A and FIG. 8B are artifacts from fat signal.

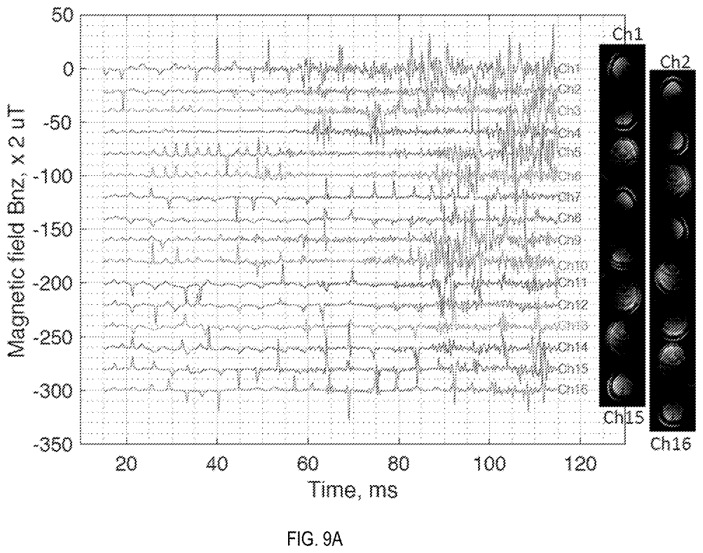

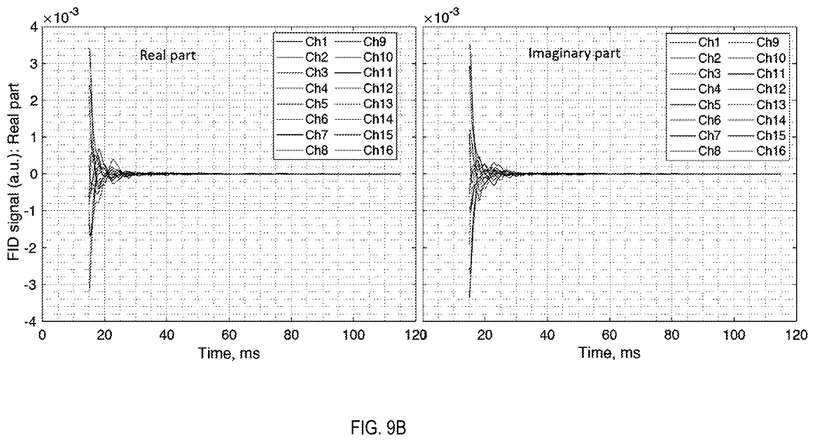

FIGS. 9A-9B show results relating to a study on a healthy subject (25-year-old male). FIG. 9A shows the resting-state qsMRI recordings and associated sensing regions in the brain. FIG. 9B shows the FID signals corresponding to the qsMRI recordings in FIG. 9A. Massive firings of action potential (~2 ms in duration) are clearly visible at multiple channels including #7 and #14 (prefrontal lobes) and #15 and #16 (occipital lobes).

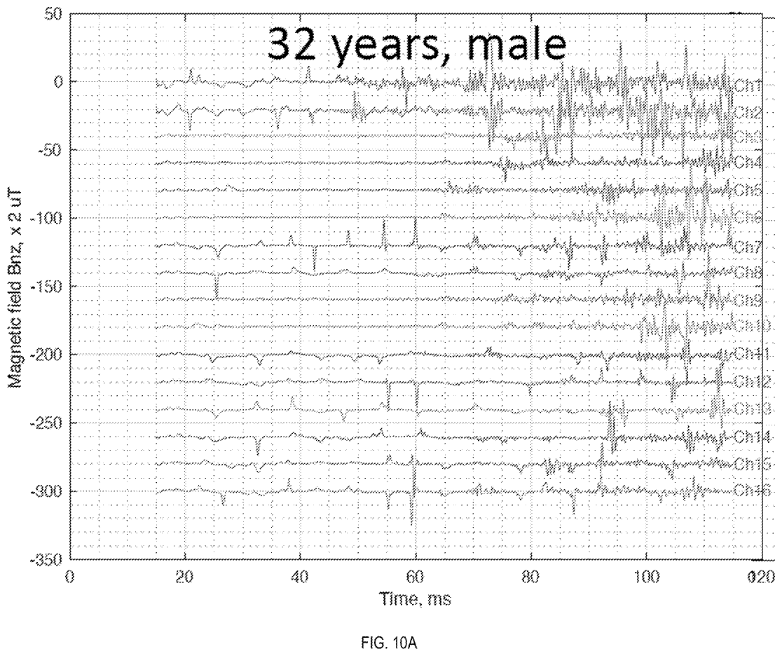

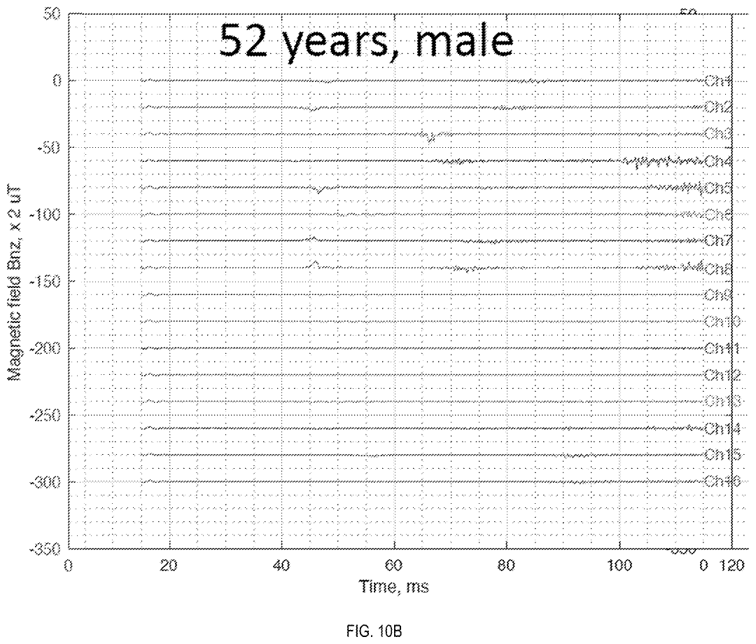

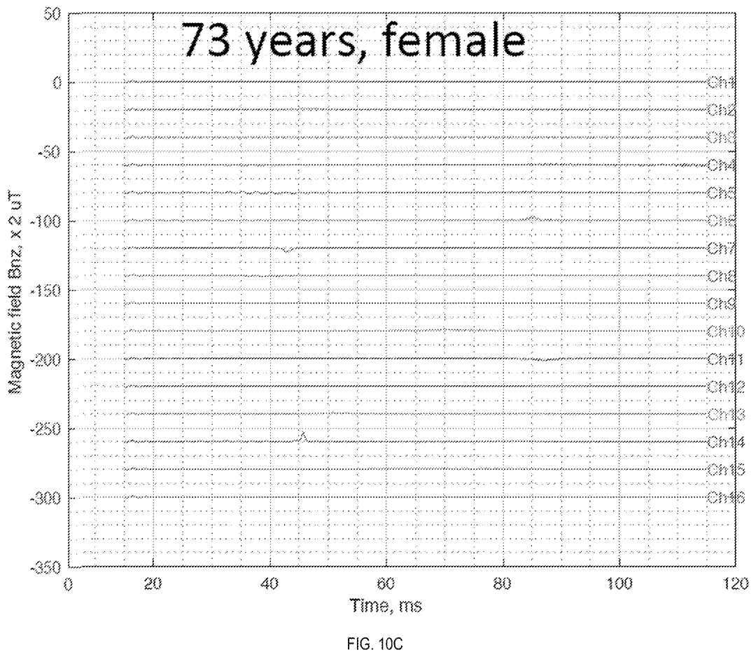

FIGS. 10A-10C show results relating to a study on healthy subjects at three different ages. The results show neuronal firings from the three cognitively-normal subjects at a resting-state MRI scan at early morning (7:00 AM): 32-year-old male (FIG. 10A), 52-year-old male (FIG. 10B), and 73-year-old female (FIG. 10C).

FIG. 11 shows a summary of neuronal firing peaks in a 100 ms-long window of FID signals from a group of 13 cognitively-normal subjects for resting-state MRI scans at early morning (7:00 AM), showing action potentials more active in awake state (self-reported) than in asleep state (self-reported) across age groups.

Functional Study on Healthy Subjects with a Finger-Tapping Task

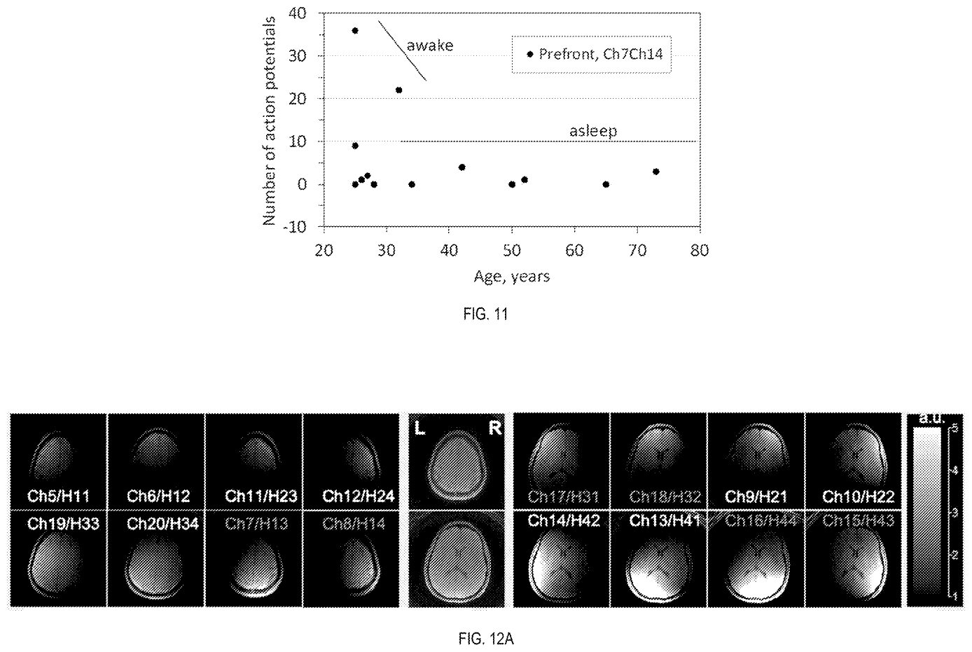

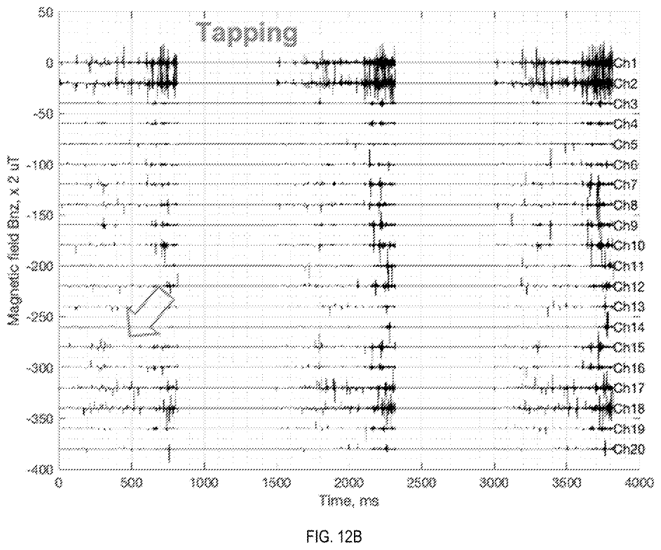

FIGS. 12A-14D illustrate results relating to studies on healthy subjects given a finger-tapping task. The studies were performed on a clinical MRI scanner at 3 T (Siemens Prisma and 20-channel Head/Neck coil). The same coil array was used for each study.

FIGS. 12A-12C show neuronal firings (the first 3 TRs) from a 43-year-old healthy female subject with and without a finger-tapping task. FIG. 12A shows MRI images of the sensing volume at individual coil elements (neck channels are not shown). Combined images (sum-of-squares) are shown in the middle. FIGS. 12B-12C show qsMRI recordings from each channel with and without the finger-tapping task. The firing peaks (shown at the arrow in FIG. 12B) are more popular (indicated with white asterisks on FIG. 12A) during the finger-tapping task than without the finger-tapping task. The neck channels have larger random noise in anterior (NE1, Ch1-2) than posterior (NE2, Ch3-4).

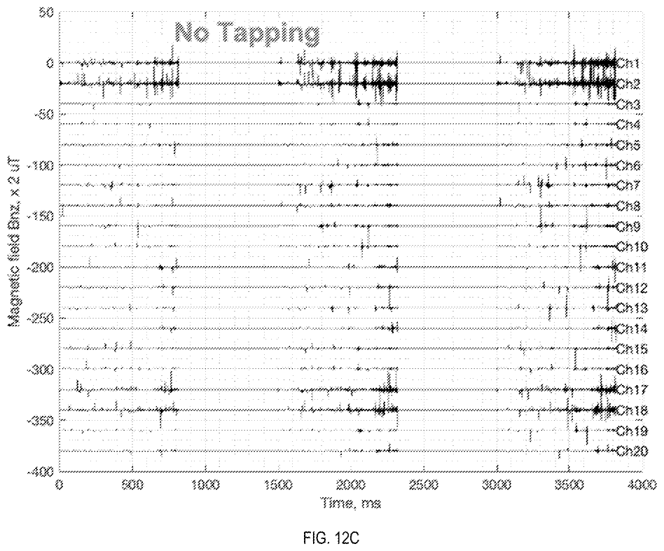

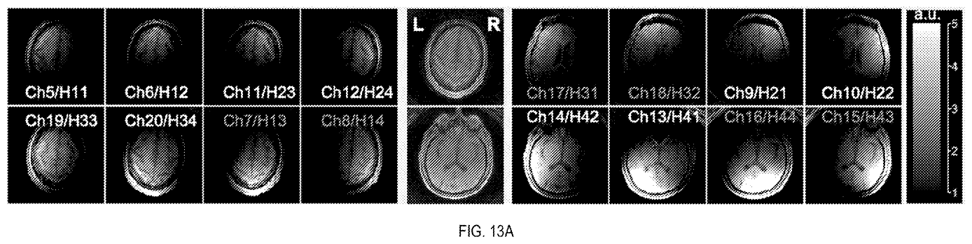

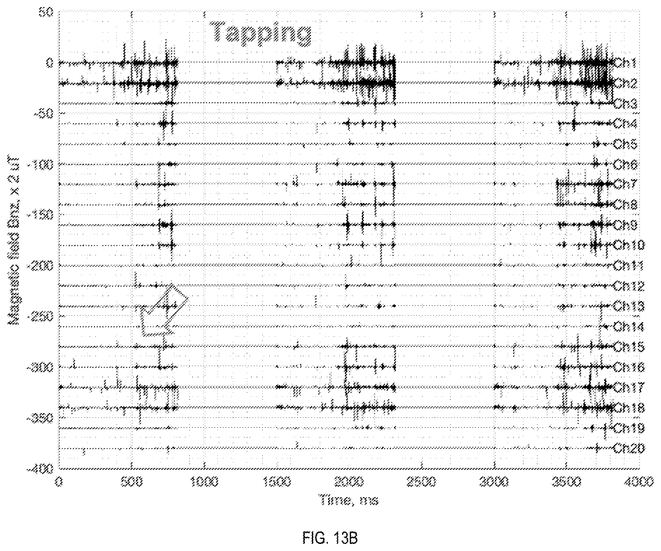

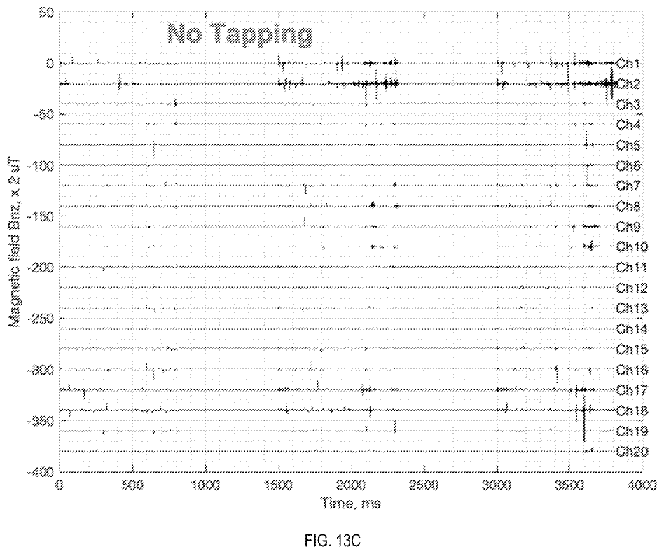

FIGS. 13A-13C show neuronal firings (the first 3 TRs) from a 74-year-old healthy male subject (normal cognition, right-handed) with and without a finger-tapping task. FIG. 13A shows MRI images of the sensing volume at individual coil elements (the neck channels are not shown). Combined images (sum-of-squares) are shown in the middle. FIGS. 13B-13C show qsMRI recordings from each channel with (FIG. 13B) and without (FIG. 13C) the finger-tapping task. The firing peaks (shown at the arrow in FIG. 13B) are more popular (indicated with white asterisks on FIG. 13A) during the finger-tapping task than without the finger-tapping task.

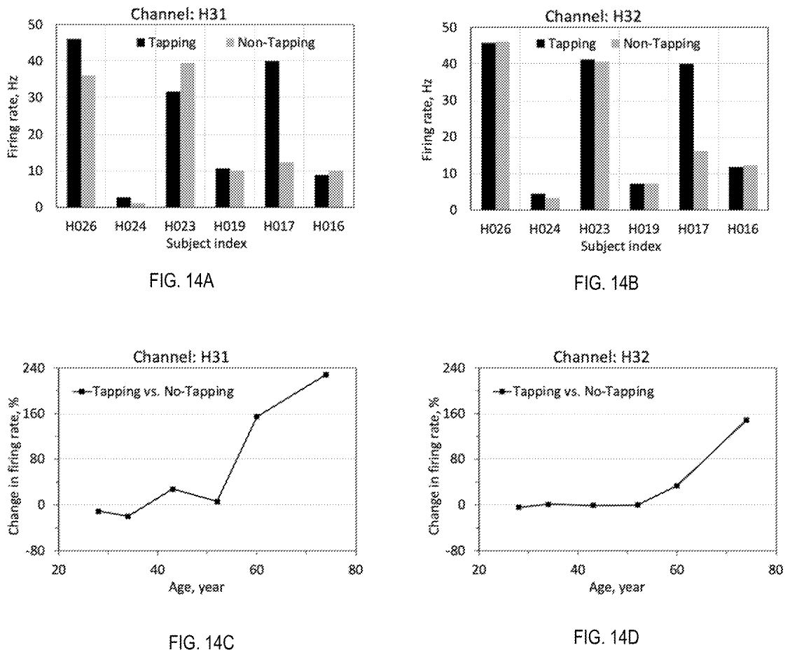

FIGS. 14A-14D shows a summary of neuronal firing rates and the changes in firing rate between with and without the finger-tapping task, in the prefrontal lobe left side (Channel H31) and in both left and right sides (Channel H32). FIGS. 14A-14B show firing rate. FIGS. 14C-14D are age-related changes in firing rate. The study subjects were right-handed, except H023 (left-handed).

### Definitions

No claim element herein is to be construed under the provisions of 35 U.S.C. § 112(f), unless the element is expressly recited using the phrase “means for.”

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

Any references herein to the positions of elements (e.g., “top,” “bottom,” “above,” “below”) are merely used to describe the orientation of various elements in the figures. It should be noted that the orientation of various elements may differ according to other exemplary embodiments, and that such variations are intended to be encompassed by the present disclosure.

Various embodiments are described in the general context of method steps, which may be implemented in one embodiment by a program product including computer-executable instructions, such as program code, executed by computers in networked environments. Generally, program modules include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps.

Software and web implementations of the present invention could be accomplished with standard programming techniques with rule-based logic and other logic to accomplish the various database searching steps, correlation steps, comparison steps and decision steps. It should also be noted that the words “component” and “module,” as used herein and in the claims, are intended to encompass implementations using one or more lines of software code, and/or hardware implementations, and/or equipment for receiving manual inputs.

As used herein, the singular forms “a,” “an,” and “the” include plural referents unless the context clearly dictates otherwise. Thus, for example, the term “a member” is intended to mean a single member or a combination of members, “a material” is intended to mean one or more materials, or a combination thereof.

As used herein, the terms “about” and “approximately” generally mean plus or minus 10% of the stated value. For example, about 0.5 would include 0.45 and 0.55, about 10 would include 9 to 11, about 1000 would include 900 to 1100.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Other substitutions, modifications, changes and omissions may also be made in the design, operating conditions and arrangement of the various exemplary embodiments without departing from the scope of the present invention.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Although the figures and description may illustrate a specific order of method steps, the order of such steps may differ from what is depicted and described, unless specified differently above. Also, two or more steps may be performed concurrently or with partial concurrence, unless specified differently above.

It is important to note that any element disclosed in one embodiment may be incorporated or utilized with any other embodiment disclosed herein. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. A system for detection of neuronal firings using quantum-sensing magnetic resonance imaging (qsMRI), comprising:
an MRI scanner, the MRI scanner programmed to execute a first pulse sequence configured for imaging and a second pulse sequence configured for free induction decay (FID) signal acquisition;
a multi-channel array coil operatively coupled to the MRI scanner, the multi-channel array coil comprising a plurality of coil elements, each coil element in the plurality of coil elements corresponding to a separate coil channel and a quantum sensing volume; and
a processor and a memory, the memory storing instructions executable by the processor, that when executed by the processor cause the processor to:
receive FID signals generated by the MRI scanner from each coil channel, the FID signals comprising a time series of samples separated by a sample time interval; determine neuronal magnetic fields induced by neuronal firings at each coil element, based on the FID signals; and
display the neuronal magnetic fields.

2. The system of claim 1, wherein the instructions further cause the processor to detect neuronal firings locally using proton nuclear spins in water molecules inside neurons as quantum sensors.

3. The system of claim 1, wherein the quantum sensing volume is defined by a coil sensitivity of each coil element or by selective excitation.

4. The system of claim 1, wherein the processor determines the neuronal magnetic fields at each coil channel by:
determining a phase of conjugate production between neighboring samples of the FID signals to determine a phase difference between the neighboring samples; and
determining neuronal magnetic fields based on the phase difference, the sample time interval, and a gyromagnetic ratio.

5. The system of claim 4, wherein the instructions further cause the processor to perform three-point smoothing or low-pass filtering in determining the phase difference and/or determining the neuronal magnetic fields based on the phase difference.

6. The system of claim 1, wherein the multi-channel array coil is a receive coil or a transceiver coil.

7. The system of claim 1, wherein the second pulse sequence is able to perform selective RF excitation and/or non-selective RF excitation.

8. The system of claim 1, wherein the MRI scanner acquires FID signals at a temporal resolution of 0.25-0.01 ms.

9. The system of claim 1, wherein each coil element corresponds to a plurality of coil channel.

10. A method of detecting neuronal firings in a subject with a magnetic resonance imaging (MRI) machine, comprising:
positioning multi-channel array coils on the subject, the multi-channel array coils comprising a plurality of coil elements;
executing an imaging pulse sequence, using the MRI machine, to acquire MRI images from each coil element in the plurality of coil elements, each coil element corresponding to a local neuronal firing volume;
executing a free induction decay (FID) signal acquisition pulse sequence, using the MRI machine, to acquire FID signals from each coil element;
locating neuronal firing sources using the acquired MRI images from each coil element;
determining neuronal magnetic fields at each coil element using the acquired FID signals based on a phase difference between neighboring samples of the acquired FID signals;
detecting neuronal firings locally using proton nuclear spins in water molecules inside neurons as endogenous quantum sensors;
modulating the neuronal magnetic fields onto the main magnetic field of the MRI machine; and
recording the neuronal firings, using the MRI machine, via the acquired FID signals from each coil element.

11. The method of claim 10, further comprising displaying neuronal firing source images and displaying the neuronal magnetic fields.

12. The method of claim 10, wherein detecting neuronal firings includes sensing the neuronal magnetic fields via perturbation of a main magnetic field provided by the MRI machine.

13. The method of claim 11, wherein displaying the neuronal magnetic fields includes providing a vertical scale showing magnetic strength and a horizontal scale for time.

14. The method of claim 10, wherein the local neuronal firing volume is passively defined by a sensitivity of each coil element.

15. The method of claim 10, wherein the local neuronal firing volume is actively defined by selective excitation of RF pulses.

16. The method of claim 10, further comprising three-point smoothing or low-pass filtering to reduce random noise interference.

17. A computer-readable medium storing instructions, which when executed by a processor, causes the processor to:
receive FID signals generated by an MRI machine, the FID signals comprising a time series of samples separated by a sampling time interval;
receive MRI images generated by the MRI machine of a sensing region;
locate neuronal firing sources, using the MRI images;
determine neuronal magnetic fields, using the FID signals, by determining a phase difference between neighboring samples of the FID signals;
utilize nuclear spins of hydrogen protons inside neurons as quantum sensors to detect neuronal firing locally;
determine the neuronal magnetic fields using the phase difference; and
display the neuronal magnetic fields with time-varying series and display neuronal firing source images.

18. The computer-readable medium of claim 17, wherein the instructions cause the processor to determine the phase difference between neighboring samples by determining a phase of conjugate production between the neighboring samples.

19. The computer-readable medium of claim 17, wherein the quantum sensors detect neuronal magnetic fields via a perturbation in a main magnetic field provided by the MRI machine.

20. The computer-readable medium of claim 17, wherein the instructions cause the processor to utilize the quantum sensors to sense electromagnetic interactions between the nuclear spins at a superposition state caused by an exciting radiofrequency (RF) pulse with a local magnetic field including a main magnetic field provided by the MRI machine and the neuronal magnetic fields.
